(19) United States
(12) Patent Application Publication
(10) Pub. No.: US 2023/0149448 A1
(43) Pub. Date: May 18, 2023

(54) METHODS FOR PREVENTING AND TREATING RESPIRATORY INFECTIONS VIA MODIFICATION OF VIRUS RECEPTOR BINDING DOMAINS USING HYPOHALOUS ACIDS

(71) Applicant: Briotech, Inc., Woodinville, WA (US)

(72) Inventors: Lori Robins, Sammamish, WA (US); Daniel James Terry, Mill Creek, WA (US); Eric Rasmussen, Kirkland, WA (US); Jeffrey Francis Williams, Langley, WA (US)

(21) Appl. No.: 17/832,263

(22) Filed: Jun. 3, 2022

Related U.S. Application Data
(60) Provisional application No. 63/196,628, filed on Jun. 3, 2021.

(51) Int. Cl. A61K 33/20; A61K 9/00; A61K 9/06; A61P 31/14

(52) U.S. Cl. CPC A61K 33/20; A61K 9/0043; A61K 9/06; A61P 31/14

(57) ABSTRACT

A method of preventing or treating respiratory virus infections is provided. The method includes administering to a subject an effective amount of a composition comprising a homogeneous hypohalous acid solution that is free of hypochlorite in HOCL solutions and free of hypobromite in HOBr solutions. The hypohalous acid modifies surface receptor binding domains of virus surface proteins and inhibits their capacity to interact with host cell receptors.

### BACKGROUND

### Technical Field

The present disclosure relates generally to the methods of prevention and treatment of respiratory virus infections in humans and animals.

### Description of the Related Art

Respiratory virus infections are responsible for high morbidity in both human and animal populations, the most notorious being those caused by the influenza myxovirus group. These infectious agents are generally transmitted through the air in the form of aerosols, often containing virus droplet nuclei that can stay suspended for many hours. Viruses in infectious droplets gain access to host cells in both the upper and lower pulmonary system by binding to epithelial cell surface membrane receptors through high affinity receptor binding domains (RBDs) on the surface of the virions. The structures of these RBDs closely resemble viruses within a class, which often bind to the same receptor but with only slight differences in the chemistry of the interactions or the mechanism of membrane transfer into the host cells. The RBDs of the various classes of respiratory viruses affecting humans and animals have been well characterized, particularly those of influenza and coronaviruses, though they differ markedly from one another.

Typically, the surface proteins and RBDs for the coronavirus class are those associated with the Severe Acute Respiratory Syndrome coronavirus 2 (SARS-CoV-2) responsible for the 2019/2020 pandemic. Mature virions initiate infection by the engagement of virus surface Spike (S) proteins with angiotensin converting enzyme-2 (ACE-2) receptors on susceptible human cells. The S protein affinity for these receptors determines tissue and cell tropism manifested during infections. The host range of this virus is dependent upon variations in S, so mutations in this protein may allow interspecies contagion. Other viruses in the class have shown similar tendencies to emerge from endemic circulation in animals and become highly contagious in human populations, e.g., SARS-CoV1, MERS virus. Vaccine development for prophylaxis in the face of the current pandemic is primarily focused on stimulating antibody and possibly other immune effector mechanisms that interfere with S protein binding to the ACE-2 receptor.

Spike protein S is a trimeric viral fusion protein shaped like a club, about 20 nm long, connected by a stalk to the membrane surrounding the virus. There are three receptor binding domain (RBD) sites on the head of the S club that vary in shapes and compositions. Depending on the state of the virus these may be shielded by glycans rather than exposed for the interaction with ACE-2 proteins. The receptor binding domains are antigenically distinct from other parts of the whole S protein, and epitopes with them are recognized by antibodies with RBD specificities. The focus on S as a vaccine immunogen is therefore aimed at preventing the virus from docking with the receptors on the membrane of susceptible cells. However, S proteins on native virus particles appear to differ from those generated by recombinant methods in having much more complex and complete patterns of glycosylation. The associated plentiful glycan structures may serve as shields interfering with access of anti-S antibodies, and subverting their potential as protective reagents.

Vaccines based on this principle of affiliating host responsive antibodies with the S proteins on the incoming virus after respiratory exposure also need to ensure that the immunoglobulin types being stimulated are able to access mucosal surfaces, rather than being restricted to submucosal interstitial fluids and systemic sites. Passively administered antibodies deposited onto mucosal surfaces in either preformed whole antibodies generated in animals, or monoclonal antibodies, even humanized ones, also run the risk of provoking host responses after repeated doses are experienced. The life span of passively administered immunoglobulin topically is not likely to be long, and this necessitates frequent dosing of an expensive solution.

### BRIEF SUMMARY

The present disclosure is directed to a method of preventing or treating severe acute respiratory virus infections including influenza, coronaviruses including SARS-CoV-2, rhinoviruses, respiratory syncytial virus, adenoviruses and other viral agents affecting the system. The method includes administering to a subject an effective amount of a composition comprising a homogeneous hypohalous acid solution, wherein the hypohalous acid modifies receptor binding domains of virus surface proteins and inhibits their capacity to interact with host cell receptors.

In some embodiments, the homogeneous hypohalous acid solution modifies the receptor binding domains of the S protein and inhibits the capacity of the receptor binding domains (RBDs) to interact with ACE2 cell receptors. The RBD of influenza and other myxoviruses are also modified by hypohalous acid solutions so as to interfere with binding to host cell receptors though both the structures of the RBD and the host cell receptors differ markedly from those involved in coronavirus infections. In other embodiments, the administering of the composition comprises administering the composition to ocular mucous epithelia. In still other embodiments, the administering of the composition comprises administering the composition topically to mucosal epithelial surfaces.

In another aspect of some embodiments, the composition is in a gel form comprising at least one viscosity-enhancing agent. In some such embodiments, at least one viscosity-enhancing agent comprises magnesium aluminum silicates, smectite clays, allophone, kaolinite, nacarite, halloysites, sodium montmorillonite, calcium montmorillonite, sauconite, vermiculite, nontronite, saponite, hectorite, bentonite, attapulgite, sepiolite, palygorskite or combinations thereof. In other embodiments the viscosity enhancing agents are polymeric derivatives of polyacrylic acid. In another aspect of some such embodiments, at least one viscosity-enhancing agent is present in the composition in an amount ranging from about 0.1 to about 10% by weight relative to the total weight of the composition.

In still another aspect of some embodiments, administering the composition comprises administering the composition in the form of droplets having a diameter in the range of from about 1 micron to about 100 microns. In some embodiments, the homogenous hypohalous acid solution is free of hypochlorite and hypobromite. In other embodiments, a total concentration of the hypochlorite and hypobromite is less than 200 ppm. In still other embodiments, the hypohalous acid is hypochlorous acid.

In yet another aspect of some embodiments, the homogenous hypohalous acid solution has a hypochlorous acid concentration from about 5 mV to about 500 mg/L, a pH from about 3.8 to about 6.5 and an ORP of about +1000 milivolts. In some embodiments, the homogenous hypohalous acid solution has a hypochlorous acid concentration from about 80 mg/L to about 300 mg/L, a pH from about 4.0 to about 4.2, and an ORP of about +1100 milivolts. In other embodiments, the homogenous hypohalous acid solution has a hypochlorous acid concentration from about 80 mg/L to about 300 mg/L, a pH from about 4.0 to about 4.2, and an ORP of about +1138 mV. In still other embodiments, the homogeneous hypohalous acid solution is an aqueous solution of hypochlorous acid having a pH from about 3.2 to about 4.9.

In one or more embodiments, the hypohalous acid is hypobromous acid. In some such embodiments, the homogenous hypohalous acid solution has a hypobromous acid concentration from about 10 mg/L to about 300 mg/L, a pH from about 4.0 to about 7.5, an oxidative reduction potential (ORP) of about +1000 millivolts. In other such embodiments, the homogenous hypohalous acid solution has a hypobromous acid concentration from about 5 mg/L to about 350 mg/L, a pH from about 4.0 to about 7.5, an oxidative reduction potential (ORP) of about +900 millivolts. In still other such embodiments, the homogeneous solution is an aqueous solution of hypobromous acid having a pH from about 5 to about 8.

In some embodiments, the administering to a subject an effective amount of a composition comprising a homogeneous hypohalous acid solution further comprises: delivering, via inhalation, aerosolized hypochlorous to the subject. In one or more other aspects, the inhalation of aerosolized hypochlorous causes generation of N-chlorotaurine (NCT) and antiviral effects. In still other aspects, the N-chlorotaurine (NCT) causes virus inactivation at sites remote from respiratory mucosae, by causing modifications that mimic modifications initiated by topical application of authentic homogeneous HOCl or HOBr.

In one or more embodiments, the homogeneous hypohalous acid solution is pure, stable, authenticated HOCl having a singular measurable peak as measured by Raman spectroscopy at 720-740 centimeters⁻¹. In other embodiments, the homogeneous hypohalous acid solution is pure, stable, authenticated HOCl having a singular measurable peak as measured by Raman spectroscopy at 728-732 centimeters⁻¹. In still other embodiments, the homogeneous hypohalous acid solution is pure, stable, authenticated HOBr having a singular measurable peak as measured by Raman spectroscopy at 615-640 centimeters⁻¹.

Another embodiment of the present disclosure is directed to a method for preventing or treating respiratory virus infections. This method includes administering to a subject an effective amount of a composition comprising a homogeneous hypohalous acid solution that is free of hypochlorite and hypobromite, wherein the hypohalous acid modifies surface receptor binding domains of virus surface proteins and inhibits their capacity to interact with host cell receptors.

In one or more embodiments, the homogeneous hypohalous acid solution modifies the receptor binding domains of the surface proteins of respiratory viruses, and inhibits the capacity of the receptor binding domains to interact with host cell receptors. In the case of SARS-CoV-2 virus the S protein is modified, and becomes unable to dock with the ACE-2 receptor. In the case of influenza virus the hemagglutinin (HA) is modified and becomes unable to bind to its corresponding receptor on human cells. In some embodiments, administering the composition comprises administering the composition to ocular mucous epithelia. In other embodiments, administering the composition comprises administering the composition topically to mucosal epithelial surfaces. In some embodiments, the respiratory virus infections comprise common cold or an influenza virus. In another aspect of some embodiments, the virus causing the common cold comprises rhinoviruses, picornaviruses or coronaviruses. In still another aspect of some embodiments, the influenza virus comprises influenza virus A, influenza virus B, or influenza virus C. In some embodiments, administering the composition includes administering the composition topically to mucosal epithelial surfaces.

In other embodiments, the composition is in a gel form comprising at least one viscosity-enhancing agent. In some such embodiments, at least one viscosity-enhancing agent comprises magnesium aluminum silicates, smectite clays, allophone, kaolinite, nacarite, halloysites, sodium montmorillonite, calcium montmorillonite, sauconite, vermiculite, nontronite, saponite, hectorite, bentonite, attapulgite, sepiolite, palygorskite or combinations thereof. In other embodiments the viscosity enhancing agents are polymeric derivatives of polyacrylic acid. In another aspect of some embodiments, at least one viscosity-enhancing agent is present in the composition in an amount ranging from about 0.1 to about 10% by weight relative to the total weight of the composition. In one or more embodiments, administering the composition includes administering the composition in the form of droplets having a diameter in the range of from about 1 micron to about 100 microns. In other embodiments, a total concentration of the hypochlorite and hypobromite is less than 200 ppm.

In still other embodiments, the hypohalous acid is hypochlorous acid. In some such embodiments, the homogenous hypohalous acid solution has a hypochlorous acid concentration from about 5 mV to about 500 mg/L, a pH from about 3.8 to about 6.5 and an ORP of about +1000 milivolts. In other such embodiments, the homogenous hypohalous acid solution has a hypochlorous acid concentration from about 80 mg/L to about 300 mg/L, a pH from about 4.0 to about 4.2, and an ORP of about +1100 milivolts. In still other such embodiments, the homogenous hypohalous acid solution has a hypochlorous acid concentration from about 80 mg/L to about 300 mg/L, a pH from about 4.0 to about 4.2, and an ORP of about +1138 mV. In yet other such embodiments, the homogeneous hypohalous acid solution is an aqueous solution of hypochlorous acid having a pH from about 3.2 to about 4.9.

In one or more another embodiments, the hypohalous acid is hypobromous acid. In some such embodiments, the homogenous hypohalous acid solution has a hypobromous acid concentration from about 10 mg/L to about 300 mg/L, a pH from about 4.0 to about 7.5, an oxidative reduction potential (ORP) of about +1000 millivolts. In other such embodiments, the homogenous hypohalous acid solution has a hypobromous acid concentration from about 5 mg/L to about 350 mg/L, a pH from about 4.0 to about 7.5, an oxidative reduction potential (ORP) of about +900 millivolts. In still other such embodiments, the homogeneous solution is an aqueous solution of hypobromous acid having a pH from about 5 to about 8.

Another embodiment of the present disclosure is directed to a system for preventing or treating respiratory virus infections in a subject. Some embodiments of this system include a liquid or a gel including a homogeneous hypohalous acid solution that is free of hypochlorite and hypobromite; and a delivery device for introducing the liquid or gel into a nasal cavity of the subject. In some embodiments, the delivery device is a syringe or a swab.

Another embodiment of the present disclosure is directed to a method of preventing or treating respiratory virus infections of human and animals, including myxoviruses, coronaviruses, severe acute respiratory syndrome coronavirus 2 (SARS-CoV-2) infections, mutated variants of SARS-CoV-2 S proteins, and other viral agents affecting the pulmonary system. The method includes: initiating generation N-chlorotaurine (NCT) which creates inhibitory effects on virus receptor binding domains (RBDs) within a subject, in response to inhalation or topical application of an effective amount of a composition comprising a homogeneous hypohalous acid solution, wherein the N-chlorotaurine (NCT) modifies receptor binding domains of virus surface proteins and inhibits their capacity to interact with host cell receptors.

In some embodiments, the generated N-chlorotaurine (NCT) causes virus inactivation at sites remote from respiratory mucosae, by causing modifications that mimic modifications initiated by topical application of authentic homogeneous HOCl or HOBr. In one or more embodiments, the homogeneous hypohalous acid solution is pure, stable, authenticated HOCl having a singular measurable peak as measured by Raman spectroscopy at 720-740 centimeters⁻¹. In other embodiments, the homogeneous hypohalous acid solution is pure, stable, authenticated HOCl having a singular measurable peak as measured by Raman spectroscopy at 728-732 centimeters⁻¹. In still other embodiments, the homogeneous hypohalous acid solution is pure, stable, authenticated HOBr having a singular measurable peak as measured by Raman spectroscopy at 615-640 centimeters⁻¹. In some such embodiments, the homogenous hypohalous acid solution is free of hypochlorite and hypobromite. In another aspect of some embodiments, the generated N-chlorotaurine (NCT) modifies the receptor binding domains of the S protein and inhibits the capacity of the receptor binding domains to interact with ACE2 cell receptors. In another aspect of some embodiments, the generated N-chlorotaurine (NCT) modifies the sialylated hemagglutinin RBD of influenza viruses and inhibits the capacity of the RBD to interact with human and animal host cell receptors.

### BRIEF DESCRIPTION OF THE SEVERAL VIEWS OF THE DRAWINGS

In the figures, identical reference numbers identify similar elements. The sizes and relative positions of elements in the figures are not necessarily drawn to scale and some of these elements are arbitrarily enlarged and positioned to improve figure legibility. Further, the particular shapes of the elements as drawn are not intended to convey any information regarding the actual shape of the particular elements, and have been solely selected for ease of recognition in the figures.

FIG. 1 is a Raman spectrum that shows pure, stable, authenticated HOCl having a singular measurable peak as measured by Raman spectroscopy at 728-732 centimeters⁻¹.

FIG. 2 shows the percentage representation of chlorine that is present as HOCl as a function of pH with substantially all available chlorine present as pure, stable, authentic HOCl at pH between 4.0-5.33.

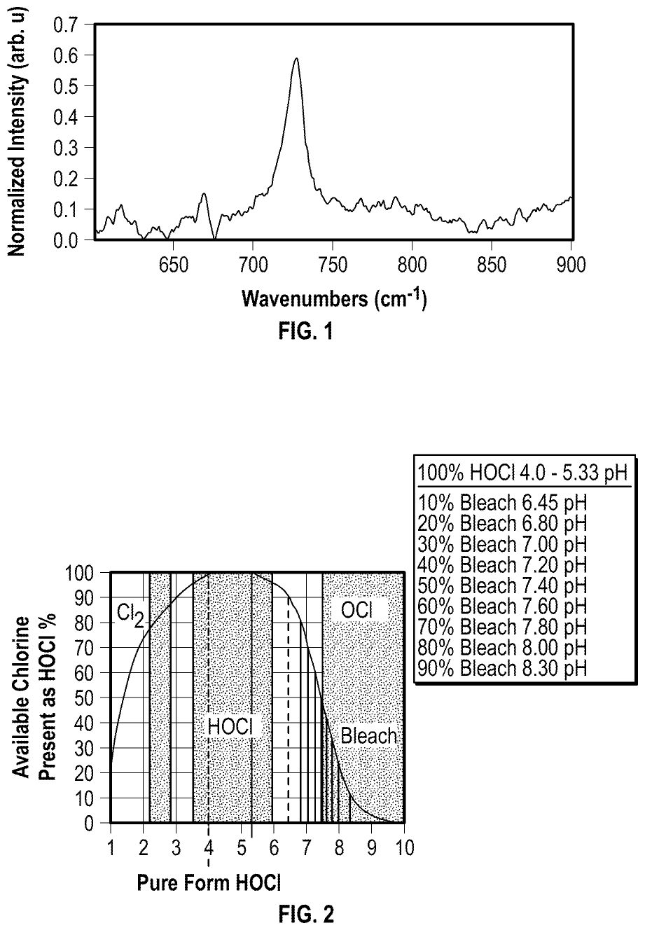

### DETAILED DESCRIPTION

Embodiments of the present disclosure use pure, safe, homogeneous aqueous solutions of hypohalous acids to rapidly inhibit the infectivity of respiratory viruses through a process that involves modification of the receptor binding domains of the virus surface components. Modification of the virion surface by the homogeneous aqueous solutions of hypohalous acids renders the virion surface unable to bind to receptors on the membranes of susceptible host cells. The virus responsible for the 2019/20 pandemic depends upon this type of binding event in order to gain access to the intracellular metabolic machinery that enables virus replication. Inhibition of the infectivity of this virus and others that depend upon similar infectivity mechanisms can be accomplished by the topical administration of homogeneous hypohalous acid solutions or viscous gels to mucous membranes in the nasal and nasopharyngeal region, or conjunctival epithelia, or by inhalation of suitably micronized droplets of solutions directly into the lower respiratory tract. From those sites there may be provision for systemic transport so as to affect virus binding to susceptible cells in non-mucosal tissues. Inhibition is effected by oxidation or halogenation of key sites on surface components that including some that are critical to the cell receptor binding process, either directly by reaction with sites in the RBDs, or by modifying neighboring structures that alter the ability of RBDs to function normally. The method of using homogeneous aqueous solutions of hypohalous acids described in the present disclosure therefore provides for utility in the prevention of respiratory virus infections in humans and animals at the points of entry. Additionally, homogeneous aqueous solutions of hypohalous acids are used adjunctively in the therapy of established infections by limiting the success of mature respiratory system virions in gaining access to other host cells within the body.

Interference with S protein binding is a target for other molecular interactions aimed at corona virus prophylaxis. Interference with the RBD of hemagglutinin of influenza viruses is the target for myxoviruses affecting the respiratory system. Small molecules are not likely to have their access to RBDs limited in a manner similar to macromolecular immunoglobulin proteins. Use of small molecules therefore provides alternative approaches to prophylaxis if small molecules were able to interact with these domains chemically so as to alter their structures or conformations, and prevent receptor binding. Hypohalous acids are capable of rapid oxidizing and halogenating interactions with many kinds of chemical entities, particularly amino acids, either singly or as parts of polypeptides and proteins. Hypohalous acids in pure form, uncontaminated with other aqueous halogen species such as hypochlorite or hypobromites, or elemental halogens, are well-tolerated, and in repeated topically administered doses are not known to be capable of host sensitization. Hypohalous acids are also not likely to lead to systemic effects from nasal mucosal deposition. As such, Hypohalous acids may form the basis of topical anti-SARS-2 prophylactic strategies, with entirely comparable application of these principles to infection prevention or treatment for other coronaviruses, and indeed all other types of respiratory viruses that afflict humans and animals. Pure hypohalous acid formulations are capable of exerting antiviral efficacy if inhaled into the alveolar spaces of the lower respiratory tract where virus-susceptible cells are known to express receptors allowing virion adherence and intracellular transfer.

Hypochlorous acid can be prepared at large scale for prophylactic and therapeutic applications in humans and animals using carefully controlled electrolysis of solutions of sodium chloride (NaCl, brine). The electrolytically generated hypochlorous acid remain homogeneous and stable for long periods, maintaining high levels of oxidative potency that allow for rapid chemical interactions with all classes of biological molecules. Modifications of macromolecules can result in the ablation of their functionalities. In this context, homogenous means that the hypochlorous acid is uniformly HOCl without containing other hypochlorites, chlorates, chlorites, molecular chlorine, buffers, or contaminants. When generated in pure and stable form HOCl is remarkably benign in its effects on tissues and physiological systems, permitting its use topically on skin and mucous membranes to control infection and enhance healing. By controlling production processes to ensure electrolytic generation of only HOCl, contamination with cytotoxic species of chlorine such as hypochlorite, chlorite, chlorate or perchlorate is avoided. This makes it possible to apply the solution or gel containing the electrolytically generated hypochlorous acid to sensitive mucosal epithelial surfaces without the hazards associated with these various contaminants.

Aerosolized hypochlorous acid can also be safely inhaled over prolonged periods as a means of delivering active hypochlorous acid to both upper and lower respiratory epithelial surfaces. From these sites there is likely delivery into the systemic circulation as a result of transmembrane mobility of hypochlorous acid, or via generation of substrate reaction products such as N-chlorotaurine (NCT) and chlorinated tyrosine or other amino acid residues. These bioactive entities show antimicrobial properties that are much longer-lived than hypochlorous acid, though they are less potent. However, they may also function as effectors of virus inactivation at sites remote from the respiratory mucosae, bringing about modifications that mimic those initiated by authentic homogeneous HOCl or HOBr applied topically. Authentic homogeneous HOBr can be generated rapidly and conveniently by conversion of HOCl using an equivalent of sodium or potassium bromide, but the conversion must be done at the point of use (POU) to avoid the intrinsic instability of this hypohalous acid.

In one aspect of the present disclosure, a homogeneous hypohalous acid solution is provided.

In some embodiments, the homogeneous hypohalous acid solution includes hypohalous acid at a concentration from about 5 mg/L to about 500 mg/L, from about 10 mg/L to about 450 mg/L, from about 50 mg/L to about 400 mg/L, from about 80 mg/L to about 300 mg/L, from about 100 mg/L to about 200 mg/L, or from about 120 mg/L to about 180 mg/L.

In some embodiments, the homogeneous hypohalous acid solution has a pH from about 3.0 to about 6.5, from about 3.0 to 5, from about 3.2 to about 4.9, from about 4.0 to about 4.2. In some embodiments, the homogeneous hypohalous acid solution has a pH of about 4.

In some embodiments, the homogeneous hypohalous acid solution has an oxidative reduction potential (ORP) from about +900 millivolts (mV) to about 1200 mV. In some embodiments, the homogeneous hypohalous acid solution has an ORP of about +1000 mV.

In other embodiments, the homogeneous hypohalous acid solution has an ORP of about +1100 mV. In still other embodiments, the homogeneous hypohalous acid solution has an ORP of about 1138 mV.

In some embodiments, the hypohalous acid is hypochlorous acid. In some of these embodiments, the homogeneous hypohalous acid solution comprises hypochlorous acid at a concentration from about 5 mg/L to about 500 mg/L, and has a pH from about 3.8 to about 6.5, and an ORP of about +1000 millivolts. In other embodiments, the homogeneous hypohalous acid solution comprises hypochlorous acid at a concentration from about 80 mg/L to about 300 mg/L, and has a pH from about 3.2 to about 4.9, and an oxidative reduction potential (ORP) of about+1100 millivolts. In further of these embodiments, the homogeneous hypohalous acid solution comprises hypochlorous acid at a concentration from about 80 mg/L to about 300 mg/L, and has a pH from about 4.0 to about 4.3, and an oxidative reduction potential (ORP) of about +1138 millivolts.

In some embodiments, the hypohalous acid is hypobromous acid. In certain of these embodiments, the homogeneous hypohalous acid solution comprises hypobromous acid at a concentration from about 10 mg/L to about 300 mg/L, and has a pH from about 4.0 to about 7.5, and an oxidative reduction potential (ORP) of about +1000 millivolts. In other of these embodiments, the homogeneous hypohalous acid solution comprises hypobromous acid at a concentration from about 5 mg/L to about 350 mg/L, and has a pH from about 4.0 to about 7.5, and an oxidative reduction potential (ORP) of about +900 millivolts.

The homogeneous hypohalous acid solution measured by Raman spectroscopy at 715-740 centimeters⁻¹, preferably at 715-732 centimeters' for HOCl, and at 615-640 centimeters' for HOBr. A singular 720-740 or 615-640 centimeters' Raman signal indicates the presence of only hypochlorous acid or only hypobromous acid (i.e., no hypochlorite or hypobromite) having a pH from about 4.0 to 7.5 and a state of isotonicity, respectively. In some embodiments, the homogeneous hypohalous acid solution includes less than 500 ppm, less than 400 ppm, less than 300 ppm, less than 200 ppm, less than 100 ppm, or less than 50 ppm hypochlorite and hypobromite. In certain embodiments, the homogeneous hypohalous acid solution includes less than 100 ppm hypochlorite and hypobromite. In other embodiments, the homogeneous hypohalous acid solution includes less than 100 ppm hypochlorite and hypobromite. In further embodiments, the homogeneous hypohalous acid solution includes less than 50 ppm hypochlorite and hypobromite.

The absence of detectable amounts of hypochlorite in HOCl solutions and hypobromite in HOBr solutions contributes to the stability of the homogeneous hypohalous acid solution by the avoidance of acceleration of reactions that degrade hypochlorous acid or hypobromous acid. Such stability relates to the primary values in hypohalous acid shelf stability in terms of the concentration of hypohalous acid in parts per million, ORP, pH and thermal tolerance from −80° C. to 100° C.

In some embodiments, the hypohalous acid is hypochlorous acid and is stable at room temperature, freezing temperatures (i.e., −80° C.) and high temperatures (i.e., 80° C.). As defined herein, stable means that the homogeneous hypochlorous acid solution described herein within a sealed container, has a detectable loss of ORP after 36 months of storage at 25° C. that is less than 10%, preferably less than 5%, and more preferably 0%. Additionally, as defined herein, stable means that the homogeneous hypochlorous acid solution described herein within a sealed impervious container, has a detectable loss of hypochlorous acid after 36 months of storage at 25° C. that is less than 50% and still more preferably less than 25%. Furthermore, as defined herein, stable means that the homogeneous hypochlorous acid described herein within a sealed impervious container, has no measurable hypochlorite or other chlorine species after 36 months of storage at 25° C.

In certain embodiments, the hypohalous acid is hypochlorous acid and the solution has a shelf life of useful inactivation efficacy up to about 36 months in a sealed impervious container. In other embodiments, the hypohalous acid is a predominantly hypobromous acid and the solution has a shelf life of useful inactivation efficacy of about four to about six hours in a sealed impervious container.

The homogeneous hypochlorous acid solution may be produced electrochemically. The electrochemical production of hypochlorous acid is carried out by treatment of a chloride-based electrolyte in a hypochlorous acid manufacturing system. Electrochemical production of a chloride-based solution is described, for example, in U.S. Application No. 63/062,287, which is hereby incorporated by reference in its entirety. In some embodiments, the homogeneous hypochlorous acid solution is produced by using precisely controlled electrolysis of a solution of sodium or potassium chloride (NaCl or KCl).

In some embodiments, the homogeneous hypobromous acid solution is provided by addition of an equimolar amount of sodium or potassium bromide to the homogenous hypochlorous acid solution, thereby converting hypochlorous acid into hypobromous acid. The complete conversion of hypochlorous acid to hypobromous acid can be detected spectrophotometrically by absorption at 250 nm. In some embodiments, the conversion of hypochlorous acid to hypobromous acid is performed at point of use. For example, hypobromous acid is prepared in situ prior to administration to patients.

In another aspect, formulation of homogeneous hypohalous acid solution is provided.

In some embodiments, the homogeneous hypohalous acid solution is formulated for topical administration. In other embodiments, the homogeneous hypohalous acid solution is formulated for administration via inhalation.

When administering topically, the homogeneous hypohalous acid solution is formulated into a gel form. The gel formulation possesses potent antimicrobial properties and is capable of interacting with biological macromolecules, such as coronavirus S proteins, sialyl hemagglutinins of influenza viruses comparable to that of an aqueous solution. Viscous formulations better adhere to mucous membranes than solutions that are formulated with certain types of aluminosilicate clays, but these require pH stabilizing buffer additives in order to maintain the conditions that optimize HOCl efficacy. In some embodiments, the buffering agent may include sodium phosphate monobasic, sodium phosphate dibasic, or combinations thereof. The pKa of HOBr enables for greater flexibility in the production of gel form for the intended uses specified in this disclosure, but the instability of this compound necessitates generation of HOBr gels at POU.

In some embodiments, the formulation is a viscous gel formulation which can better adhere to mucous membranes than the solution. In some embodiments, the gel is applied to a human or animal using syringe or a swab.

In some embodiments, the gel formulation comprises a homogeneous hypohalous acid solution of the present disclosure. The homogenous hypohalous acid solution may be a homogenous hypochlorous acid solution or a homogenous hypobromous acid solution. In some embodiments, the homogeneous hypohalous acid solution is present in the formulation in an amount ranging from about 0.005 to 0.1% by weight relative to the total weight of the formulation.

The gel formulation further includes at least one viscosity-enhancing agent. The term “viscosity-enhancing agent” refers to any agent that, when applied in various concentrations in an aqueous medium, results in the formation of stable hydrogels that exhibit thixotropic properties. The at least one viscosity-enhancing agent can be chosen from natural clay and synthetic clay. In an embodiment, the hydrogel viscosity can be achieved by the use of an entirely synthetic mineral which is akin to the natural clay mineral hectorite in structure and composition. Unlike natural clay, a synthetic mineral is typically free of impurities yet can be equal in structure to natural hectorite. Examples of the viscosity-enhancing agent include, but are not limited to, magnesium aluminum silicates, smectite clays, and an amorphous clay mineral, such as allophone; two-layer type crystalline clay minerals, such as equidimensional crystal, kaolinite, and nacarite; elongate crystals, such as halloysites; three-layer type crystalline clay minerals, such as sodium montmorillonite, calcium montmorillonite, sauconite, vermiculite, nontronite, saponite, hectorite, and bentonite; chain structure crystalline clay minerals, such as attapulgite, sepiolite, and palygorskite; or mixtures thereof. In some embodiments, aluminum silicate is used as the viscosity-enhancing agent. In some embodiments polymeric derivatives of polyacrylic acid are used as viscosity enhancing agents.

In some embodiments, the at least one viscosity-enhancing agent is present in the formulation in an amount ranging from about 0.1 to about 10% by weight relative to the total weight of the formulation.

In some embodiments, the homogeneous hypohalous acid solution of the present disclosure is formulated for the aerosol delivery by inhalation of aerosolized solution of homogenous hypohalous acid. When the homogenous hypohalous acid solution is administered by aerosolization, it is preferably administered in the form of droplets having a diameter in the range of from about 1 micron to about 100 microns.

The aerosolization of the homogeneous hypohalous acid solution of the present disclosure can be performed using, for example, piezoelectric nebulizers, spray bottles, metered spray pumps, metered-dose inhalers, and bag-on valve spray cans.

Regardless of the types of formulations of hypohalous acids being applied, it is found that alterations in the capacity of those segments of respiratory viruses responsible for binding to host cell receptors occur upon exposure to the hypohalous acid formulations. The changes can be demonstrated by the use of antibody reagents that have specificity for RBD epitopes. For example, after exposure of S protein of SARS-CoV-2 to hypohalous acid formulations there is a significant reduction in the amount of remaining S protein that can be bound by immobilized RBD specific antibody. This reduction may be due to direct modification of the RBD region of S protein, or be due to modification of other components of the protein that then influence availability of specific RBD epitopes to antibodies directed against them. These changes may be compositional or conformational, but there is direct evidence from mass spectrometry of modifications to amino acid residues on S protein that are consistent with the effects of hypohalous acid exposure. Such changes occurring within the RBDs are responsible for the effects on binding to ACE2.

By inhibiting the interactions between the S protein RBDs and the complementary sites on the ACE2 protein receptors on the membranes of susceptible cells, hypohalous acid formulations of the present disclosure enable the introduction of entirely novel approaches to the prevention and treatment of COVID-19 disease. Since similar structures and mechanisms are involved in the adherence to and entry into host cells of respiratory viruses in general, including influenza viruses, rhinoviruses and other coronaviruses, exposure to hypohalous acid formulations is likely due to a result in similar outcomes as with SARS-Cov-2 virus. Influenza virus particles are extremely susceptible to chlorine in the form of a mixture of hypochlorous acid and hypochlorite ions, and the RBD of influenza viruses is modified by chlorine dioxide and becomes unable to bind to its host cell receptor. If RBDs are modified in the ways identified herein then infectious viral particles are denied access to the internalization process that enables them to commandeer those metabolic processes required for virus replication. This inhibition has implications for the avoidance of destructive pathological events that normally follow successful infection. Topical application of hypohalous acid formulations described herein to nasal mucosal surfaces, for example, are used in an effective use pattern before exposure of individuals to potential infectious virus environments so as to prevent viral adherence to susceptible epithelial cells.

Additionally, a use pattern is devised for prevention of respiratory virus infection after such exposure, taking advantage of the known protracted time course of adherence and cell penetration by all these classes of viruses. During the several hours over which these processes take place in the case of SARS-CoV-2 virions penetration of the S protein regions and ongoing alterations in the RBDs, hypohalous acid molecules in these formulations interfere sufficiently to provide a protective benefit. Introduction of hypohalous acid formulations suitable for ocular tissue application by sprayer or via instillation of droplets reduces the risks of virus infection via exposure of conjunctival and corneal epithelial cells. Those mucosae are known to express high levels of ACE2 receptors used by coronaviruses. Similar use patterns can be expected to be effective in domestic animals, particularly household pets, which are known to acquire SARS-CoV-2 infections from infected human subjects.

The inhalation of finely microaerosolized preparations from the homogeneous hypohalous acid solution enables the arrival of these compounds in the alveoli of the lower respiratory system, where cell surface receptors are abundant on alveolar epithelial cells. Use of the hypohalous acid formulations of the present disclosure offers a means of preventing infection of these cells, and the avoidance of initiation of the COVID19 disease process, and, for example, of influenza virus-initiated pulmonary infections. In established infections, viral replication and release lead to subsequent infection of neighboring cells by the same mechanisms of interaction of RBDs and local cell surface receptors. Therefore, the presence of hypohalous acid inhibitors provides adjunctive support to other therapeutic interventions in the treatment of patients and animals suffering from respiratory virus diseases.

While the evidence from examples illustrated below supports the conclusion that hypohalous acid formulations in vitro are capable of direct inhibitory effects on RBDs, it is clear that hypohalous acids in the in vivo environment quickly give rise to biologically active products of their reactivity with naturally occurring substrates such as N chloro taurine (NCT), and others. NCT is known to be powerfully antiviral in vivo, and capable of modifications of proteins in ways entirely comparable to those resulting from HOCl and HOBr exposure. It is likely therefore that the beneficial effects of these hypohalous acid formulations in the body after topical or inhalation applications may come about from the continuing presence of NCT and other altered natural substrates, and their effects on virus RBDs. Some substrates may enter the systemic circulation and give rise to these inhibitory processes at tissue sites other than ectodermally derived epithelial cells, since surface receptors are widely distributed in other organs and on internal endodermally or mesodermally derived cell surfaces.

The evidence from examples detailed below indicates that the routes of exposure envisioned for deployment of the present disclosure are compatible with safe delivery of the formulations of homogeneous hypohalous acids that bring about effective inhibition of virus RBD. They will therefore create no undue risks to human subjects or animals when administered in these ways either prophylactically or therapeutically.

### EXAMPLES

### Example 1: Preparation of Pure Stable Briotech Hypochlorous Acid

Hypochlorous acid solutions useful in the methods of the present disclosure, BrioHOCL™, was supplied by Briotech Inc., Woodinville, Wash. Briefly, HOCl results from electrolysis of an aqueous solution of sodium chloride so as to provide at the anode conditions that attract and stabilize reaction products that form HOCl. The end-product is a solution with a range of pH on packaging and storage of 3.8-4.5 at warehouse environmental temperatures (3.5° C. to 35° C.), an ORP of +1100 my, a salt (NaCl) concentration of either 0.85% or 1.8-2% by weight, and a free chlorine concentration of 250-300 mg/L at the time of production. No adjustments are ever made to this HOCl solution by the addition of buffers, metal ions, organic heterocyclic halogen stabilizers or pH modifiers of any sort, at any level.

As described herein, the pure homogeneous aqueous hypochlorous acid produced by the HOCl manufacturing system and method is defined as a free chlorine concentration solution of hypochlorous acid that does not contain stabilizing buffers and does not contain detectable hypochlorite, and in which the pH is measured in the spectrum that completes its chemical reaction and at a spectrographic range of 720-740 centimeters' with a pH that maximizes its ORP. In this regard, authentic pure HOCl is defined as a HOCl mixture with no amount of hypochlorites, mixed oxidants, or other contaminants.

Any amount of hypochlorite that exists in a less than authentic, unadulterated impure HOCl solution (known scientifically as “mixed oxidant”), creates a condition of reactivity that drives the mixed oxidant HOCl solution into a degrading chemical reaction which eventually leads to a full hypochlorite state. This degrading chemical reaction in a mixed oxidant HOCl solution has been typically been contained in prior systems through use of stabilizing buffers. For this reason, mixed oxidant HOCl solution can be identified as such (i.e., a less than authentic, unadulterated pure HOCl solution), even if they claim to be “pure,” by their inclusion of stabilizing buffers, hypochlorite, or both. Even a very small amount of either stabilizing buffers, hypochlorite, or both renders any such solution as a mixed oxidant, and not an authentic, unadulterated pure aqueous hypochlorous acid. Furthermore, the addition of stabilizing buffers adulterates any solution into an impure state by definition

Convenient conversion of these solutions of HOCl to HOBr was accomplished by the addition of equimolar quantities of sodium bromide (NaBr) to HOCl, resulting in complete conversion to HOBr detectable spectrophotometrically by a new absorption peak at 250 nm.

### Example 2: Inactivation of SARS-CoV-2 Virus Infectivity by Aqueous Briotech Hypochlorous Acid

This experiment was done to establish that HOCl solutions inactivate the intact mature infectious SARS-2 corona virus. The experiment was performed at the National Microbiology Laboratory of the Public Health Agency of Canada in Winnipeg, Manitoba, Canada.

Stocks of an isolated SARS-CoV-2 were propagated in Vero E6 cells using the TCID50 Median Tissue Culture infectious assay in a 96 well plate format. For inactivation assays the viruses were concentrated by ultracentrifugation. Virus inocula were exposed to Briotech HOCl for 10 minutes; control suspensions were exposed to phosphate buffered saline. Five replicates were used for each condition and the experiment was repeated three times. Treatment conditions consisted of exposing suspensions to the HOCl solution and control solutions in 2 mL vials by adding 50 microliters of virus, and vortexing to ensure thorough mixing, followed by incubation for ten minutes at room temperature.

At incubation, 900 microliters of sterile 1% sodium thiosulfate was added to quench the HOCl. Controlled experiments established that this quenching process had no effect on virus infectivity. Vial contents were titrated by serial dilution into Dulbecco's MEM medium plus 2% fetal bovine serum, and then added to semiconfluent monolayers of Vero E6 cells for endpoint titration by TCID50 assay, using conventional incubation conditions at 37C in 5% CO2 for sufficient time for virus plaque enumeration. Corona virus suspensions exposed to HOCl at 165 ppm of titratable Cl showed an average reduction in titer of 99.96%.

The results show that Briotech HOCl is capable of inactivation of infectious SARS-CoV-2 virions to a high level after ten minutes of exposure.

### Example 3: Inactivation of SARS-CoV-2 Virus Infectivity after Short Exposure

This experiment was done to establish that HOCl solutions inactivate the intact mature infectious SARS-CoV-2 corona virus after very short periods of exposure. The experiment was performed at the Human Microbiology Institute, 101 Avenue of the Americas, New York, N.Y.

Stocks of an isolate of SARS-CoV-2 were propagated in Vero 76 cells using MEM culture medium with 2% fetal bovine serum plus 50 microgram/mL of gentamycin. Briotech HOCl at 180 ppm of titratable Cl was used to inactivate samples of virus suspensions exposed for 120 seconds, compared to virus recoveries from aliquots exposed to culture medium alone, or to 45% ethanol as a positive control. Exposures were conducted at room temperature and quenching of HOCl was accomplished by addition of 10× amounts of medium containing fetal bovine serum. Exposures under each condition were performed in triplicate.

Virus recoveries were determined by TCID50 end point dilution assays, using 10 fold serial dilutions into 80-90% confluent monolayers of Vero 76 cells. Inoculated 96 well plates were incubated at 37° C. in CO2 incubators for 6 days prior to scoring. Control preparations were included to ensure that the quenching process adequately inactivated the infectious virus particles and that no continuing inactivation by HOCl was observed after quenching.

Corona virus suspensions exposed to HOCl for 120 seconds showed average reductions in infectivity of 99.92%. After ten minutes of exposure, the positive control exposed to 45% ethanol showed average reductions of 99.98%.

The results showed that Briotech HOCl is capable of inactivation of infectious SARS-CoV-2 corona virus to a high level after only 120 seconds of exposure.

### Example 4: Exposure of SARS-CoV-2 Spike Protein to HOCl or HOBr

Ability of HOCl or HOBr to modify the receptor binding domain (RBD) of Spike S protein is demonstrated by exposure of Spike S protein to either Briotech HOCl or HOBr.

This protocol was performed in 96 well plate format using two different immunoreagents both with specificity for RBD on the S protein (Sourced from Cusabio Technology LLC). The first antibody was used to coat the wells and served as the capture reagent upon subsequent exposure to a solution of S protein. After washing away excess unbound S, the detector antibody-HRP conjugate was applied with specificity for RBD. Upon addition of a HRP substrate this detector antibody conjugate generated a chromophore, the intensity of which was proportional to the amount of S captured. Color changes were detected spectrophotometrically. Suitable controls included quenching of the hypohalous acids at the end of the exposure time (5 minutes) to ensure that there were no artifactual effects of HOCl on the components of the assay that might influence S binding.

The results show that after exposure to HOCl or HOBr at 150 ppm the amount of S protein remaining was very significantly reduced, so that there was complete inhibition of chromophore formation. These findings demonstrate that alterations in the capacity of S protein to bind to RBD specific antibodies resulted from exposure of S protein to solutions of hypohalous acids.

### Example 5: Inhibition of ACE2 Receptor Binding by S Protein after Exposure to Hypohalous Acid

Ability of HOCl or HOBr to inhibit S protein from binding to ACE2 receptor protein is demonstrated by exposure of S protein to HOCl or HOBr.

RayBio Covid 19 Spike ACE2 binding assay kit was used in a 96 well format wherein the plate wells were coated with recombinantly expressed ACE2. The test detects binding of recombinant S protein to the immobilized ACE2 by use of an enzyme-linked antibody specific to S. After free S is removed by washing, this HRP conjugated antibody is added in the presence of a trimethylbenzidine substrate, generating a blue color, the intensity of which is proportional to the amount of S complexed with the ACE2. Exposure of S protein to HOCl or HOBr prior to addition to the plate wells enables demonstration of inhibition of the capacity of S to interact with ACE2 receptor protein.

### Example 6: Safety of Pure Hypohalous Acid Upon Exposure of Intact Skin and Respiratory Mucous Membranes, and after Exposure Orally or by Inhalation in Experimental Animals

Experimental exposures of rodents were done according to OECD method 423 for acute oral toxicity assessment, OECD method 434 for acute dermal toxicity and OECD method 433 for acute inhalation. Results are tabulated below:

TABLE 1

| System | Organism | Duration | Behavior | Histopathology | Source | Date |
|---|---|---|---|---|---|---|
| Dermal | Mammal | 14 days | No | No | Baltic | July, |
| | (rat) | | change | pathology | Control | 2020 |
| | | | | | Laboratories | |
| Respiratory | Mammal | 4 hours of | No | No | Stillmeadow | January, |
| | (rat) | exposure, | change | pathology | Laboratories, | 2021 |
| | | evaluated | | | Texas | |
| | | at 14 days | | | | |
| Gastrointestinal | Mammal | one time | No | No | Stillmeadow | July, |
| | (rat) | exposure | change | pathology | Laboratories, | 2020 |
| | | to 5000 | | | Texas | |
| | | mg/kg of | | | | |
| | | aqueous | | | | |
| | | product, | | | | |
| | | evaluated | | | | |
| | | at 14 days | | | | |

The results show that exposure of animals to pure stable HOCl of the present disclosure induces no detectable pathological changes by any of the tested routes.

### Example 7: Safety of Hypohalous Acid Upon Exposure of Human Subjects Via Inhalation

The safety of exposure of human subjects to microaerosolized pure stable HOCl of the present disclosure via the respiratory route was evaluated.

Human volunteers were exposed to dense microaerosols of HOCl for periods of 2-5 minutes and submitted subjective reports of the outcomes of these experiences. Data on a total of 400 such episodes were collected under medical supervision. No serious adverse effects were recorded, and minor complaints (nose irritation, slight impact on ease of deep breathing) were limited to approximately 3%, which disappeared upon cessation of exposure to HOCl.

The results support the safety of human subject exposure for brief periods to pure HOCl by the respiratory route.

### Example 8: Safety of HOCl Gel Upon Exposure of Human Subjects to Untranasal Administration of Hypohalous Acid Gel Preparations

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of preventing or treating severe acute respiratory syndrome coronavirus 2 (SARS-CoV-2) infections, the method comprising administering to a subject an effective amount of a composition comprising a homogeneous hypohalous acid solution, wherein the hypohalous acid modifies S protein receptor binding domains of virus surface proteins of SARS-CoV-2 and mutated variants of SARS-Cov-2, and inhibits their capacity to interact with host cell receptors.

2. A method of preventing or treating respiratory virus infections of human and animals, the method comprising administering to a subject an effective amount of a composition comprising a homogeneous hypohalous acid solution, wherein the hypohalous acid modifies surface components of respiratory viruses of humans and animals and inhibits their capacity to interact with host cell receptors.

3. The method of claim 1 wherein the administering to the subject the effective amount of the composition is via inhalation of an aerosolized solution of the composition.

4. (canceled)

5. The method of claim 1, wherein administering the composition comprises administering the composition to mucous epithelia.

6. (canceled)

7. The method of claim 1, wherein the composition is in a gel form comprising at least one viscosity-enhancing agent present in an amount ranging from 0.1 to 10% by weight relative to the total weight of the composition.

8. The method of claim 1, wherein the at least one viscosity-enhancing agent comprises magnesium aluminum silicates, smectite clays, allophone, kaolinite, nacarite, halloysites, sodium montmorillonite, calcium montmorillonite, sauconite, vermiculite, nontronite, saponite, hectorite, bentonite, attapulgite, sepiolite, palygorskite, or combinations thereof.

9. (canceled)

10. The method of claim 1, wherein administering the composition comprises administering the composition in the form of droplets having a diameter in the range of from about 1 micron to about 100 microns.

11. (canceled)

12. (canceled)

13. (canceled)

14. The method of claim 1, wherein the homogenous hypohalous acid solution has a hypochlorous acid concentration from about 5 mV to about 500 mg/L, a pH from about 3.8 to about 6.5, an ORP of about +1000 milivolts, and is free of hypochlorite.

15. The method of claim 1, wherein the homogenous hypohalous acid solution has a hypochlorous acid concentration from about 80 mg/L to about 300 mg/L, a pH from about 4.0 to about 4.2, and an ORP of about +1100 milivolts, and is free of hypobromite.

16.-60. (canceled)

61. The method of claim 2, wherein administering the composition comprises administering the composition topically to mucous epithelia.

62. The method of claim 2, wherein the composition is in a gel form comprising at least one viscosity-enhancing agent present in an amount ranging from 0.1 to 10% by weight relative to the total weight of the composition.

63. The method of claim 2, wherein the at least one viscosity-enhancing agent comprises magnesium aluminum silicates, smectite clays, allophone, kaolinite, nacarite, halloysites, sodium montmorillonite, calcium montmorillonite, sauconite, vermiculite, nontronite, saponite, hectorite, bentonite, attapulgite, sepiolite, palygorskite, or combinations thereof.

64. The method of claim 2, wherein administering the composition comprises administering the composition in the form of droplets having a diameter in the range of from about 1 micron to about 100 microns.

65. The method of claim 2, wherein the homogenous hypohalous acid solution has a hypochlorous acid concentration from about 5 mg/mL to about 500 mg/L, a pH from about 3.8 to about 6.5, and an ORP of about +1000 millivolts, and is free of hypochlorite.

66. The method of claim 2, wherein the homogenous hypohalous acid solution has a hypobromous acid concentration from about 5 mg/L to about 350 mg/L, a pH from about 4.0 to about 7.5, an oxidative reduction potential (ORP) of about +1000 millivolt, and is free of hypobromite.

67. The method of claim 2, wherein the administering to the subject the effective amount of the composition is via inhalation of an aerosolized solution of the composition.

68. A system for preventing or treating respiratory virus infections in a subject, comprising:
a liquid or a gel including a homogeneous hypohalous acid solution that is free of hypochlorite and hypobromite; and
a delivery device for introducing the liquid or gel into a nasal cavity of the subject.

69. The system of claim 68, wherein the delivery device is a syringe or a swab.

70. The system of claim 68, wherein the homogeneous hypohalous acid solution is administered to a via inhalation of an aerosolized solution of the homogeneous hypohalous acid solution.
